(19) United States
(12) Patent Application Publication
(10) Pub. No.: US 2015/0064063 A1
(43) Pub. Date: Mar. 5, 2015

(54) APPARATUSES AND METHODS FOR CLEANING AND DRYING PROSTHETIC LINERS

(71) Applicant: Aaron Fitzsimmons, Nashville, TN (US)

(72) Inventor: Aaron Fitzsimmons, Nashville, TN (US)

(21) Appl. No.: 14/014,627

(22) Filed: Aug. 30, 2013

(51) Int. Cl. A61L 2/10; A61L 2/08

(52) U.S. Cl. CPC A61L 2/10; A61L 2/084
USPC 422/24; 250/454.11; 422/22

(57) ABSTRACT

Apparatuses are provided that eliminate microbes in a prosthetic liner. Apparatuses are provided that eliminate odors and moisture from a prosthetic liner. Apparatuses and methods disclosed illuminate prosthetic liners with anti-microbial lights and push air past the prosthetic liners.

### FIELD

The present disclosure relates generally to prosthetic liners. In exemplary though non-limiting embodiments, the present disclosure relates to apparatuses and methods to clean and dry prosthetic liners.

### BACKGROUND

A prosthetic is an artificial device used in medicine to replace a body part lost through injury, disease or congenital conditions. Prosthetic devices may be divided into types of prosthesis and further divided by anatomical region. Limb prostheses include upper extremity and lower extremity prostheses. Each of these can be further divided based upon the level of amputation and/or loss of body part.

Over the past ten years, the medical field has made numerous improvements, including in the field of prosthetics. Better surgery techniques and advanced components of prosthetic legs, for example, have allowed amputees to accomplish feats previously unattainable under earlier technologies. Amputees are able to lead more active and athletic lives than ever before.

One area of improvement for prosthetics is in socket technology. A prosthetic socket joins a patient's residual limb to the prosthesis. A well-designed socket ensures adhesion and proper fit of the prosthesis. Under current technologies, prosthetic sockets are typically custom-made for each patient based upon the shape and condition of the residual limb and mobility grade. Modern artificial limbs usually include a liner. The liner acts as a second skin between the moveable soft tissue of the residual limb and a hard surface of the prosthetic. Liners are typically made of a relatively soft material and may provide better suction between the residual limb and a prosthetic than without a liner. Liners increase safety and comfort, while protecting the skin of the residual limb by reducing friction and pressure points. Liners may be gel based liners.

Because of the various improvements in prosthetics, leading to increased activity among amputees, problems may develop with gel liner conditions after substantial use of a prosthetic device. For example, bacteria and odor often become embedded into the gel liners. Also, patients may have troubles with liners becoming sweaty after wearing a prosthetic for substantial periods of time. Unfortunately, cleaning a liner with normal soap and water can be very difficult because liners are generally porous. Because of these factors, gel liners may have decreased lifespans and replacements may cost anywhere from $300 to $1000 depending on the type of liner.

Accordingly, there is need for new apparatuses and methods that clean and dry a prosthetic liner without damaging the liner with additional wear and tear.

### SUMMARY

In an embodiment of the present invention, an anti-microbial prosthetic liner stand is provided, having: a base having a fan mounted thereon; a tube extending vertically above the base; a light socket attached to an upper portion of the tube and extending vertically along of the tube; and a receiving knob attached to a top portion of the tube. The receiving knob is configured to receive a prosthetic liner such that the liner rests upon the receiving knob and extends downward around at least a portion of the tube. An anti-microbial light bulb is installed in the light socket. The stand is configured to illuminate at least a portion of the prosthetic liner with light from the anti-microbial light bulb when the prosthetic liner rests upon the receiving knob. The fan is configured to push air vertically upward past at least a portion of the prosthetic liner when the prosthetic liner rests upon the receiving knob.

The anti-microbial light bulb may emit one of ultraviolet light and blue light. The light socket may be approximately nine inches in length. The anti-microbial light bulb may be a nine inch, 254 nm germicidal ultraviolet light bulb. The anti-microbial light bulb may emit light having a wavelength of approximately 405 nm to approximately 470 nm. The stand may further include housing contained within a bottom portion of the tube. The stand may further include a ballast and a regulator contained within the housing. The tube may have a length of approximately twenty inches. The tube may have a diameter of approximately 0.36 inches. The stand may further include wiring passing within an interior of the tube down to the base and connecting both the light socket and the fan to a power source.

In an embodiment of the present invention, an apparatus is provided, having: a base; a tube extending vertically above the base; and a light socket attached to an upper portion of the tube. An anti-microbial light bulb is installed in the light socket. The tube is configured to receive a prosthetic liner such that at least a portion of the prosthetic liner is illuminated by light from the anti-microbial light bulb. The base comprises a fan configured to push air vertically past at least a portion of the prosthetic liner.

The anti-microbial light bulb may emit one of ultraviolet light and blue light. The light socket may be approximately nine inches in length. The anti-microbial light bulb may be a nine inch, 254 nm germicidal ultraviolet light bulb. The anti-microbial light bulb may emit light having a wavelength of approximately 405 nm to approximately 470 nm.

In an embodiment of the present invention, a method to clean and dry a prosthetic liner is provided, including: illuminating the prosthetic liner with an anti-microbial light bulb; and pushing air across a surface of the prosthetic liner. The prosthetic liner is placed on a tube having the anti-microbial light bulb installed thereon. The tube extends vertically above a based having a fan installed thereon. The fan is configured to push air upwards along the tube.

The anti-microbial light bulb may emit one of ultraviolet light and blue light. The light socket may be approximately nine inches in length. The anti-microbial light bulb may be a nine inch, 254 nm germicidal ultraviolet light bulb. The anti-microbial light bulb may emit light having a wavelength of approximately 405 nm to approximately 470 nm.

### DESCRIPTION OF DRAWINGS

FIG. 1 is a front view of an apparatus according to an exemplary embodiment of the present invention.

FIG. 2 is a top view of a portion of an apparatus according to an exemplary embodiment of the present invention.

FIG. 3 is a front view of the apparatus shown in FIG. 1.

FIG. 4 is a top view of the portion of the apparatus shown in FIG. 2.

FIG. 5 is a front view of an anti-microbial prosthetic liner stand with an prosthetic liner placed thereon according to an exemplary embodiment of the present invention.

### DESCRIPTION

Like reference characters denote like parts in the drawings.

Embodiments of the present disclosure provide apparatuses and methods to clean and dry prosthetic liners. Embodiments of the present disclosure may illuminate prosthetic liners with an anti-microbial light source or bulb. Embodiments of the present disclosure may illuminate prosthetic liners with an ultraviolet light source, which may be a 254 nm germicidal ultraviolet light bulb. Embodiments of the present disclosure may illuminate prosthetic liners with blue light, which may have a wavelength of approximately 405 nm to approximately 470 nm. Embodiments of the present disclosure may incorporate a fan configured to push air past prosthetic liners, which fan may facilitate drying of the prosthetic liners and/or control temperatures. Embodiments of the present disclosure include methods of cleaning and drying prosthetic liners using anti-microbial light and a fan installed upon a stand.

Embodiments of the present disclosure allow prosthetic liners to be cleaned without soap and water. Embodiments of the present disclosure provide anti-microbial treatment to prosthetic liners without submitted the prosthetic liners to additional wear and tear. Embodiments of the present disclosure prevent moisture buildup in prosthetic liners, providing cleaner liners and odor elimination.

Microbes may be destroyed or eliminated using light-based anti-microbial approaches. Ultraviolet (UV) light may be used to kill microbes, including bacteria, by breaking down DNA of the microbes. Blue light, particularly in a wavelength range of approximately 405 nm to approximately 470 nm, also has anti-microbial properties. Blue light may be used instead of UV light in applications involving potential UV radiation of a patient.

Embodiments of the present disclosure include an anti-microbial light bulb, which may emit UV or blue light, to kill microbes that have become embedded and/or attached to prosthetic liners. Embodiments of the present disclosure include a nine inch, 254 nm germicidal light bulb installed in a light socket attached to tube. Other embodiments of the present disclosure include an anti-microbial light bulb emitting blue light installed in the light socket. The tube may extend vertically from a base, which base may incorporate a fan. The fan may be aligned such that it blows air upwards along the tube. The tube may include a receiving knob attached to a top of the tube, which may be configured to receive a prosthetic liner. When placed upon the receiving knob, the prosthetic liner may extend downward along the tube, including all or a portion of a length of the tube having the light socket attached thereon. Embodiments of the present disclosure may include a stand having the tube extending vertically from the base having a fan on a top portion of the base aligned to blow air towards the receiving knob.

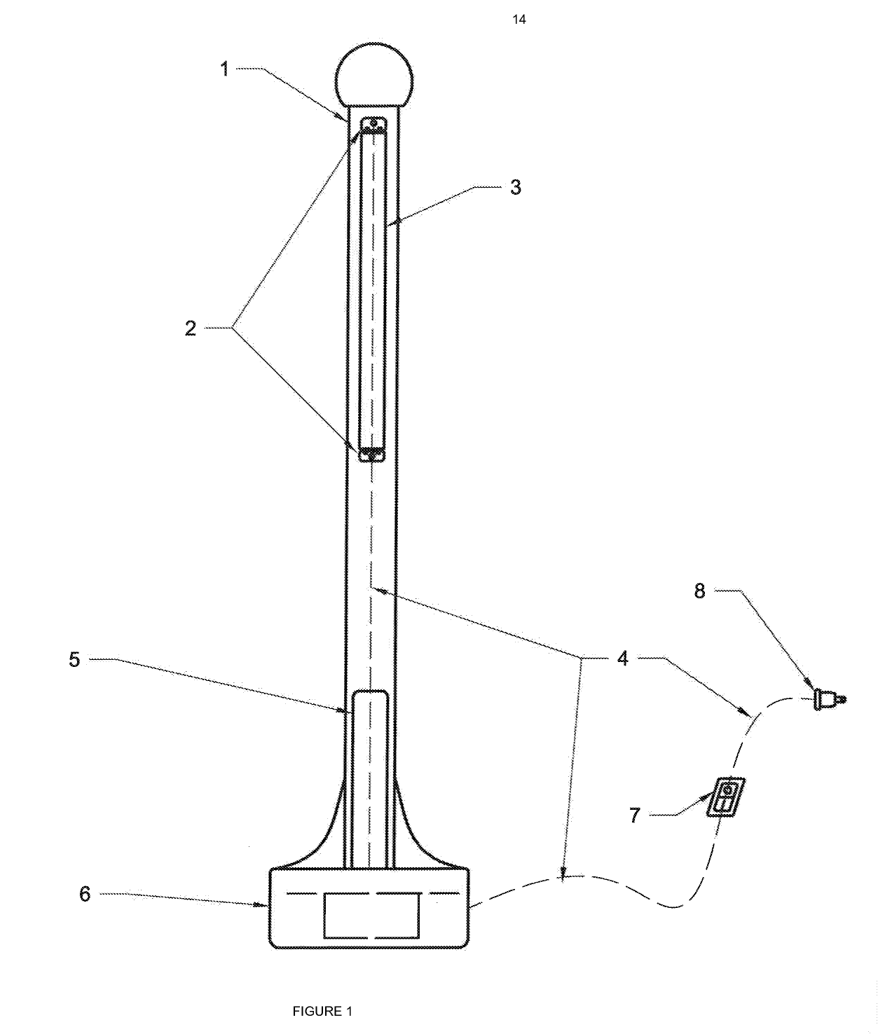

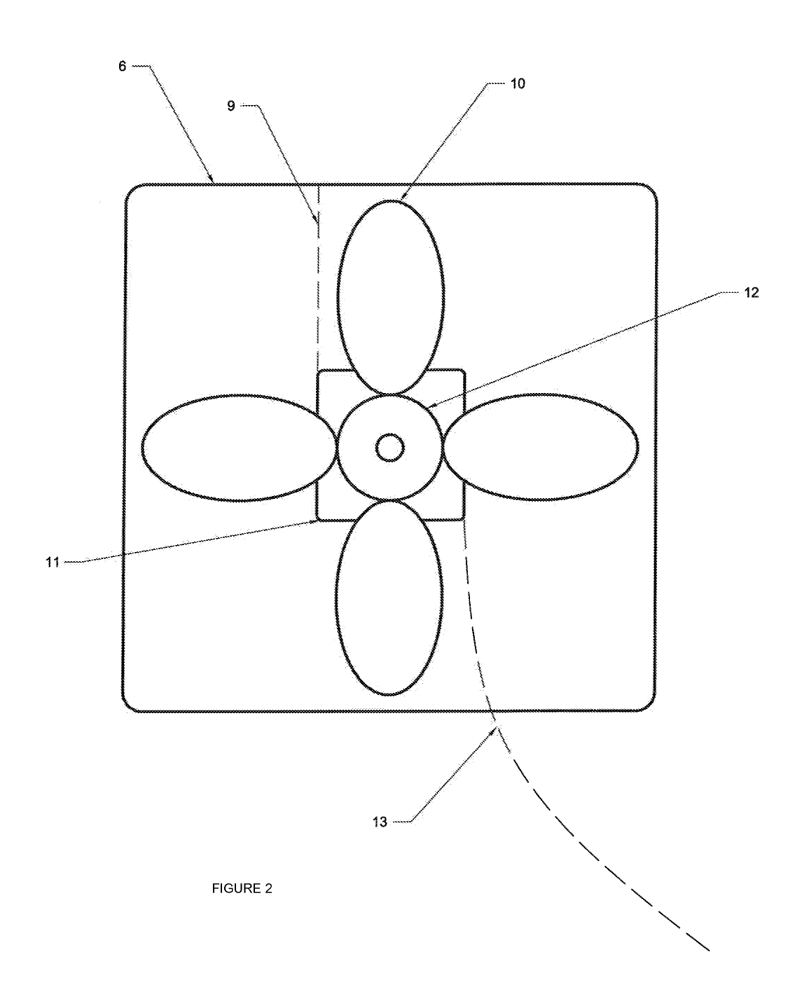

Referring now to FIG. 1, which shows a front view of an exemplary embodiment of the present disclosure, an anti-microbial prosthetic liner stand is provided having base 6, which includes housing for a fan. FIG. 2 provides a top view of an embodiment of base 6 showing fan motor 11 for operation of a fan, a plurality of fan blades 10 above a top surface of base 6, and axis 12 allowing rotation of the plurality of fan blades 10. Though FIG. 2 shows an embodiment having four fan blades, embodiments may have more or less than four fan blades 10. Plurality of fan blades 10 are arranged such that they may push air vertically upward along tube 1 when rotated by fan motor 11. See, e.g., FIG. 1.

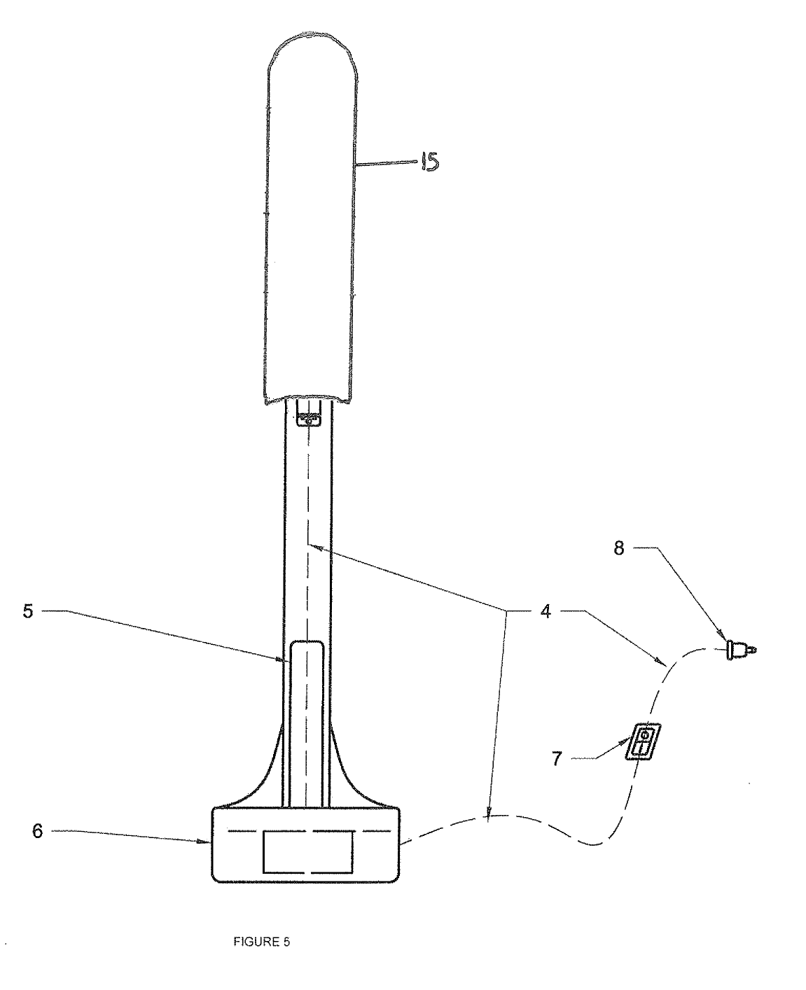

As shown in the embodiment in FIG. 1, tube 1 may extend vertically from a center portion of base 10. On an upper portion of tube 1, a light socket 2 may be incorporated, which may be configured to receive anti-microbial light bulb 3. Anti-microbial light bulb 3 may emit UV light or blue light. The embodiment shown in FIG. 1 includes receiving knob 14. Prosthetic liner 15 may be placed over tube 1 such that it rests upon receiving knob 14, partially covering tube 1 and partially or entirely covering anti-microbial light bulb 3. See, e.g., FIG. 5.

The embodiment shown in FIG. 1 includes wire 4 passing along an interior of tube 1. Wire 4 may connect light socket 2 and fan motor 11 to an on/off switch 7 and to a power source via plug 8, which is insertable into a standard outlet. Housing 5 may be incorporated inside tube 1 on a bottom portion of tube 1 and extending vertically above base 6. Housing 5 may include a ballast and a regulator (not shown). Wiring connections for the light socket 2 and the fan motor 11 may be placed within housing 5. Wiring from the fan motor 11 to housing 5 may include wire portion 9. See, e.g., FIG. 2. Wiring from the fan motor 11 to on/off switch 7 may include second wire portion 13. See, e.g., FIG. 2.

By eliminate or killing microbes with anti-microbial light exposure rather than subjected the prosthetic liners to chemicals, solvents, and/or mechanical cleaning forces, damage to the prosthetic liners may be minimized. The anti-microbial light treatment is effective at substantially eliminating microbes embedded in or on the prosthetic liners. Additionally, effectively drying the prosthetic liners with a fan ensures that moisture will be removed from the prosthetic liners.

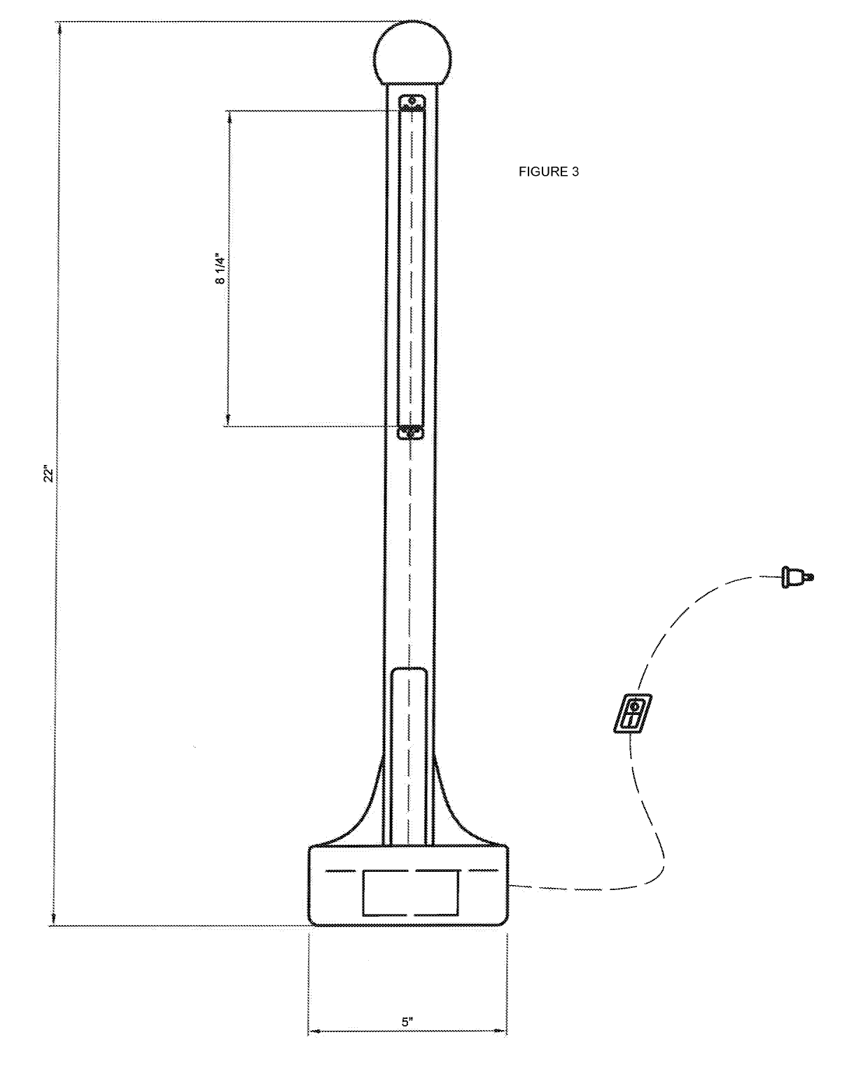

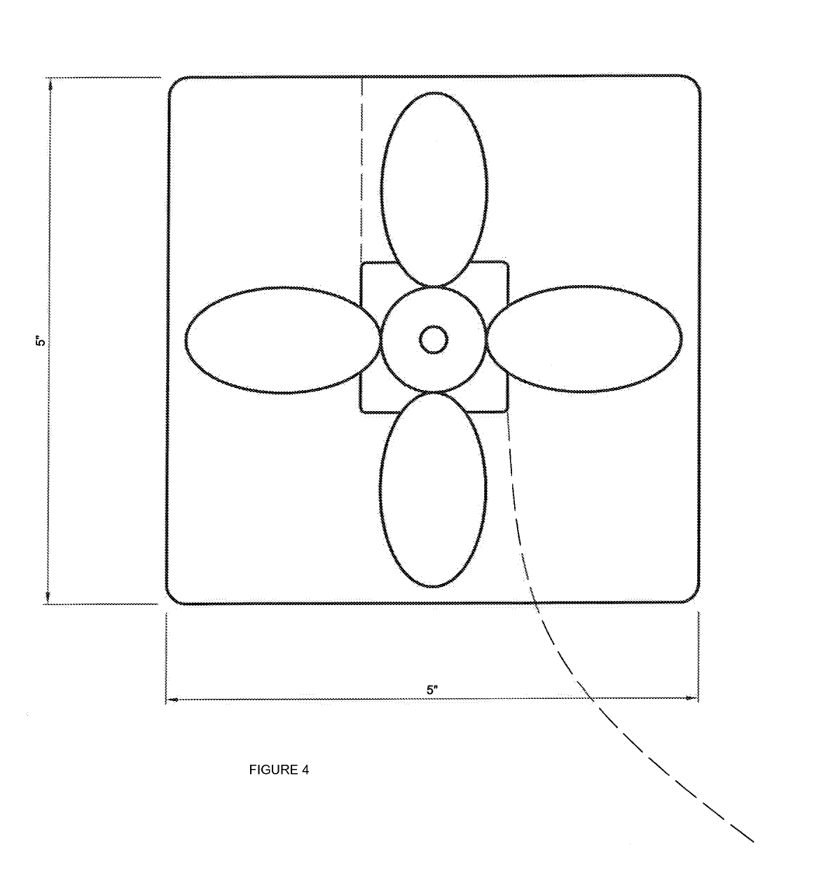

FIGS. 3 and 4 show various dimensions for exemplary embodiments of the present disclosure. Embodiments of the present disclosure may have a total height of approximately twenty-two inches and may have a tube 1 having a height of approximately twenty inches. Tube 1 may have a radius of approximately 0.36 inches. Embodiments of the present disclosure have a substantially square base having dimensions of approximately five inches by approximately five inches. Embodiments may incorporate base structures that are approximately rectangular or any other shape.

While the embodiments are described with reference to various implementations and exploitations, it will be understood that these embodiments are illustrative and that the scope of the inventions is not limited to them. Many variations, modifications, additions, and improvements are possible. Further still, any steps described herein may be carried out in any desired order, and any desired steps may be added or deleted. The specification and drawings are accordingly to be regarded in an illustrative rather than in a restrictive sense.

What is claimed is:

1. An anti-microbial prosthetic liner stand, comprising:
a base having a fan mounted thereon;
a tube extending vertically above the base;
a light socket attached to an upper portion of the tube and extending vertically along of the tube; and
a receiving knob attached to a top portion of the tube;
wherein the receiving knob is configured to receive a prosthetic liner such that the liner rests upon the receiving knob and extends downward around at least a portion of the tube;
wherein an anti-microbial light bulb is installed in the light socket;
wherein the stand is configured to illuminate at least a portion of the prosthetic liner with light from the anti-microbial light bulb when the prosthetic liner rests upon the receiving knob; and
wherein the fan is configured to push air vertically upward past at least a portion of the prosthetic liner when the prosthetic liner rests upon the receiving knob.

2. The stand of claim 1, wherein the anti-microbial light bulb emits one of ultraviolet light and blue light.

3. The stand of claim 1, wherein the light socket is approximately nine inches in length.

4. The stand of claim 3, wherein the anti-microbial light bulb is a nine inch, 254 nm germicidal ultraviolet light bulb.

5. The stand of claim 2, wherein the anti-microbial light bulb emits light having a wavelength of approximately 405 nm to approximately 470 nm.

6. The stand of claim 1, further comprising: housing contained within a bottom portion of the tube.

7. The stand of claim 6, further comprising: a ballast and a regulator contained within the housing.

8. The stand of claim 1, wherein the tube has a length of approximately twenty inches.

9. The stand of claim 1, wherein the tube has a diameter of approximately 0.36 inches.

10. The stand of claim 7, further comprising: wiring passing within an interior of the tube down to the base and connecting both the light socket and the fan to a power source.

11. An apparatus, comprising:
a base;
a tube extending vertically above the base;
a light socket attached to an upper portion of the tube;
wherein an anti-microbial light bulb is installed in the light socket;
wherein the tube is configured to receive a prosthetic liner such that at least a portion of the prosthetic liner is illuminated by light from the anti-microbial light bulb; and
wherein the base comprises a fan configured to push air vertically past at least a portion of the prosthetic liner.

12. The apparatus of claim 11, wherein the anti-microbial light bulb emits one of ultraviolet light and blue light.

13. The apparatus of claim 11, wherein the light socket is approximately nine inches in length.

14. The apparatus of claim 13, wherein the anti-microbial light bulb is a nine inch, 254 nm germicidal ultraviolet light bulb.

15. The apparatus of claim 12, wherein the anti-microbial light bulb emits light having a wavelength of approximately 405 nm to approximately 470 nm.

16. A method to clean and dry a prosthetic liner, comprising:
illuminating the prosthetic liner with an anti-microbial light bulb; and
pushing air across a surface of the prosthetic liner;
wherein the prosthetic liner is placed on a tube having the anti-microbial light bulb installed thereon;
wherein the tube extends vertically above a based having a fan installed thereon; and
wherein the fan is configured to push air upwards along the tube.

17. The method of claim 16, wherein the anti-microbial light bulb emits one of ultraviolet light and blue light.

18. The method of claim 17, wherein the anti-microbial light bulb is a 254 nm germicidal ultraviolet light bulb.

19. The method of claim 17, wherein the anti-microbial light bulb emits light having a wavelength of approximately 405 nm to approximately 470 nm.
